(19) United States
(12) Patent Application Publication
(10) Pub. No.: US 2022/0104629 A1
(43) Pub. Date: Apr. 7, 2022

(54) Leg Pillow

(71) Applicant: James J. LESKO, Pittsburgh, PA (US)

(72) Inventor: James J. LESKO, Pittsburgh, PA (US)

(21) Appl. No.: 17/491,719

(22) Filed: Oct. 1, 2021

Related U.S. Application Data
(60) Provisional application No. 63/086,144, filed on Oct. 1, 2020.

(51) Int. Cl. A47C 20/00; A61F 7/08

(52) U.S. Cl. CPC A47C 20/021; A61F 2007/0043; A61F 7/08

(57) ABSTRACT

A leg pillow is provided that includes a pad having a band or a strap connected thereto. The band allows the pillow to be secured to the user's leg. The pillow can be worn by a user during sleep or at other times, and aids in reducing heat loss in the user's legs and separating the user's legs, which improve circulation and reduce joint pain.

### CROSS-REFERENCE TO RELATED APPLICATIONS

The present application claims the benefit of U.S. Provisional Patent Application No. 63/086,144 filed on Oct. 1, 2020, which is herein incorporated by reference in its entirety.

### BACKGROUND OF THE INVENTION

The present application is directed to a leg pillow comprising a band on one side to hold the leg pillow securely to the lower leg (i.e., below the knee) throughout the sleeping period at night.

Unattached pillows, often used to try to eliminate the problems of lower leg pain, heat loss and poor blood circulation are easily dislocated with typical twisting movements while sleeping; and often become a nuisance, rather than helping undisturbed sleep. Bulky, electric heating pads can be used to provide heat to lower legs, as another option. But electric heating pads carry potential safety hazards and messy wires, and often cause too much burning heat, which has the opposite effect of loss of sleep. The bulky thermal mass type heating pads lose heat over time and become a nuisance, restricting leg movement. Heavy, bulky socks are another alternative but do not allow a normal comfortable loss of heat, causing sweating and discomfort.

### SUMMARY OF THE INVENTION

The present application provides for an insulating leg pillow, which retains normal body heat. It is an elegant solution to eliminate the pain in the lower legs, caused by greater heat loss and/or often poor blood circulation. There is no need for the bulky heating pads previously described.

The leg pillow of the present application can achieve several purposes and address several shortcomings of the prior art, including: (1) eliminating pressure points where one lower leg crosses the other leg while an individual is sleeping on their side (bone on bone); (2) Significantly reducing or eliminating joint stress of the hip and knee, by keeping a more comfortable, normal distance between the legs while sleeping on the side; (3) Retaining body heat in the legs, to eliminate cold in the lower legs. The lower legs lose body heat because the body mass (fat) is less than in other parts of the body. This greater proportional heat loss combined with poor blood circulation causes lower leg pain and/or cramps.

In accordance with a first aspect of the present application, an apparatus is provided. The apparatus includes a pillow, and a band having two opposing ends each affixed to the pillow, the band at least partially overlying a surface of the pillow, wherein the band is configured to be pulled away from the surface of the pillow to provide a passage formed between the band and the surface of the pillow, and wherein the passage is configured to receive a leg of a user and the band secures the pillow to the leg of the user.

Implementations of the apparatus may include one or more of the following features, separately or in combination. In certain embodiments of the apparatus, the band can be formed from an elastic material configured to be expandable away from the surface of the pillow to allow the user to insert his or her leg into the passage, and to contract back towards the surface of the pillow when released to secure the pillow to the leg of the user. In various embodiments, the band may include a plurality of spaced apart and substantially parallel bands.

The pillow may include an outer layer and a padded material arranged within the outer layer. The band and the outer layer of the pillow may each be made of a cotton material. The band may have a length of approximately twelve inches and the pillow may have a length of approximately eleven inches, and the passage formed perpendicularly to the length of the pillow. A height of the pillow may be approximately nineteen inches and a width of the pillow may be approximately six inches.

The pillow may include two opposing edges and each of two ends of the bands may be attached to one of the two opposing edges. In certain embodiments, each of the two ends of the band are sewn to one of the two opposing edges of the pillow. In other embodiments, a first end of the band is sewn to a first opposing edge of the pillow and a second end of the band is attached to a second opposing edge of the pillow by a detachable connection means. The detachable connection means may include one or more of: a button and opening slot to receive the button, a tab and tab receiving arrangement, a zipper mechanism, hook and loop fasteners, a strap and loop configured to receive the strap therethrough with strap securing mechanism, a string or other tying mechanism, a snap and button arrangement, and magnetic connectors.

The apparatus is configured to be worn by a user during sleep and is configured to warm the user's leg during sleep and increase space between the user's legs during sleep. The band of the apparatus is configured to enable the user to rotate the pillow around the leg between different positions.

In accordance with a further aspect of the present application, a method for reducing leg pain and heat loss of a user is provided. The method comprises providing a leg pillow to the user, the leg pillow comprising a pillow; and a band having two opposing ends each affixed to the pillow, the band at least partially overlying a surface of the pillow, wherein the band is configured to be pulled away from the surface of the pillow to provide a passage formed between the band and the surface of the pillow. The method further comprises inserting a leg of the user through the passage to secure the pillow to the leg of the user. The method may further comprise inserting the leg of a user through the passage to secure the pillow prior to the user sleeping, and the pillow is secured to the leg of the user during sleep.

### BRIEF DESCRIPTION OF THE FIGURES

FIG. 1A shows a rear side view of a leg pillow according to an embodiment of the present application.

FIG. 1B shows a side view of a leg pillow according to an embodiment of the present application.

FIG. 1C shows a perspective view of a leg pillow according to an embodiment of the present application.

FIG. 2A shows a side view of the leg pillow according to an embodiment of the present application secured to a user's leg.

FIG. 2B shows a front view of the leg pillow according to an embodiment of the present application secured to a user's leg.

### DETAILED DESCRIPTION OF THE FIGURES

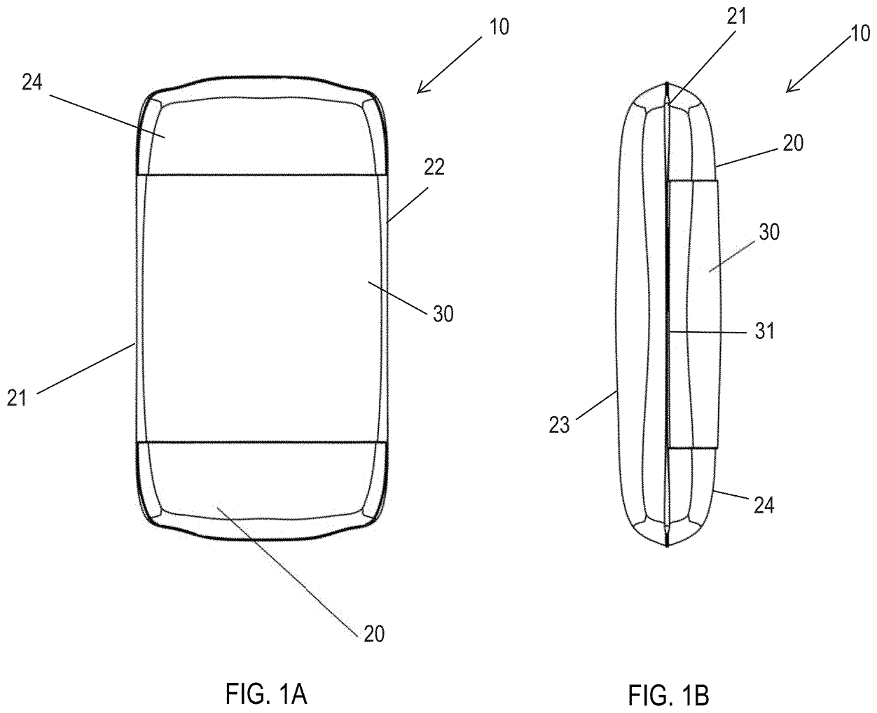

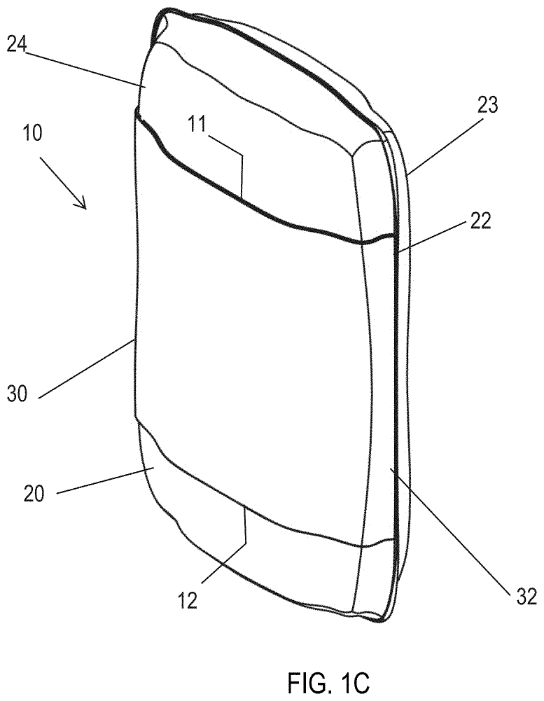

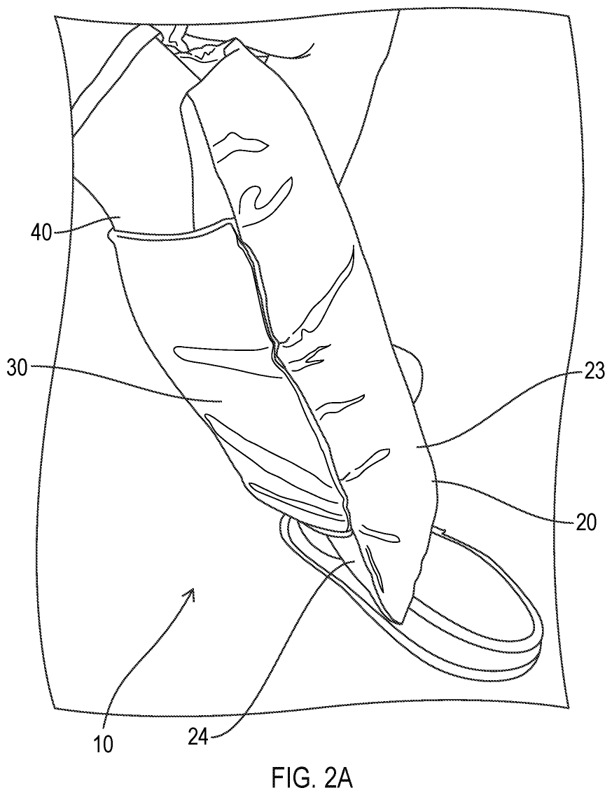

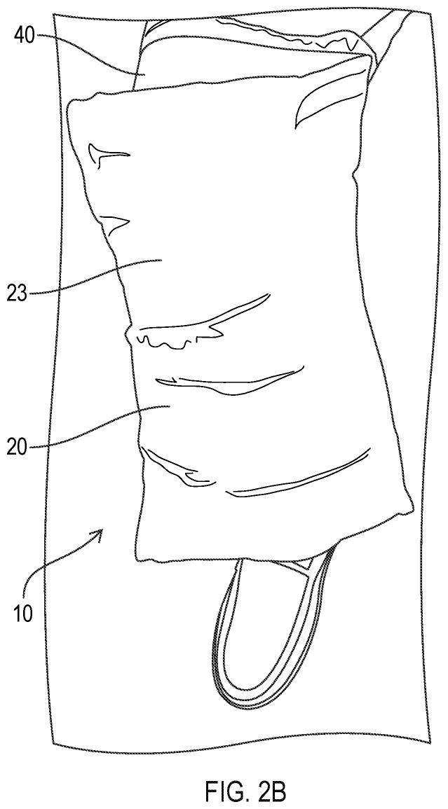

Illustrative embodiments of the leg pillow 10 of the present application are shown in FIGS. 1A-2B. FIGS. 1A-1C show rear, side and perspective views, respectively, of an embodiment of a leg pillow 10 of the present application. FIGS. 2A-2B show various views of the use of a leg pillow 10 according to an embodiment of the present application.

The leg pillow 10 of the present application comprises a pad 20 and one or more bands or straps 30 that are provided to secure the pillow or pad 20 to the user's leg 40. The pillow 20 includes a front side 23 and a rear side 24, and on the sides between the front side 23 and rear side 24 includes a first edge 21 and a second edge 22. The band 30 includes a first end 31 and a second end 32, the first end 31 being secured to the first edge 21 of the pillow 20 and the second end 31 being secured to the second edge 22 of the pillow 20. The ends 31, 32 of the band are secured to the edges 21, 22 of the pillow 20, respectively, by sewing them together, or by an alternative detachable connection means that allows the band 30 to be detached from the pillow 20 at least on one end 31, 32. The detachable connection means may be arranged on the band 30 and/or the pillow 20 interchangeably for their respective interaction and engagement, and can include, but are not limited to, a button and opening slot to receive the button, a tab and tab receiving arrangement, a zipper mechanism, hook and loop fasteners, a strap and loop configured to receive the strap therethrough with strap securing mechanism, a string, laces, or other tying mechanism, a snap and button arrangement, magnetic connectors, and any of the above-mentioned components in combination or duplicate.

In the example embodiment shown in FIGS. 1A-1C, the pillow 20 has approximate dimensions of nineteen inches by eleven inches by six inches (height by length by width) with an approximately twelve-inch-long band 30 centered on the reverse side 24 along the length of the of the leg pad 20. However, the leg pillow 10 is not limited to these dimensions, and alternative sizes and shapes can be provided without departing from the scope of the invention. The band 30 is sewn or otherwise attached along two opposing edges 21, 22 of the leg pad 20. An opening 11 between the band 30 and the rear side 24 of the pad 20 is provided for the user to insert his or her leg 40 through a passage and exit through a second opening 12 between the band 30 and the rear side 24 of the pad 20. The band 30 used to hold the leg pillow 10 in position enables the pad 20 to be used to eliminate pressure points on the lower leg 40 while sleeping.

The leg pad 20 of the leg pillow 10 can be filled with a soft foam, or any other soft material or padded material suitable for use in a pillow and is covered by an outer layer of any suitable fabric material, which can include for example a woven cotton material. The band 30 can be made of the same or similar cloth or fabric material. In other embodiments, the band 30 can be made of an elastic material to expand and contract around the user's leg 40. The band 30 may also include one or more bands or straps spaced apart, rather than a single wide and elongated band.

While trying to eliminate pressure points and joint stress, the insulating effect of the leg pillow 10 also provides important benefits because of the uniform body heat throughout the night, and in the morning waking-up with comfortable warm legs.

Use of only one leg pillow 10 is required, as one leg pillow 10 is effective for both legs in a cross-leg position or parallel. During sleep, the leg pillow 10 is non-intrusive on the user and it does not have to be removed during the night to make short necessary ventures. While being worn by the user, the leg pillow 10 is configured to be rotatable around the user's leg 40. The band 30 of the leg pillow 10 allows the user to easily rotate the leg pillow 10 around the leg 40 to a new position without losing control of the leg pillow 10.

While there have been shown and described and pointed out fundamental novel features of the invention as applied to preferred embodiments thereof, it will be understood that various omissions and substitutions and changes in the form and details of the devices and methods described may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice.

What is claimed is:

1. An apparatus comprising:
a pillow; and
a band having two opposing ends each affixed to the pillow, the band at least partially overlying a surface of the pillow,
wherein the band is configured to be pulled away from the surface of the pillow to provide a passage formed between the band and the surface of the pillow; and
wherein the passage is configured to receive a leg of a user and the band secures the pillow to the leg of the user.

2. The apparatus according to claim 1, wherein the band is formed from an elastic material configured to be expandable away from the surface of the pillow to allow the user to insert his or her leg into the passage, and to contract back towards the surface of the pillow when released to secure the pillow to the leg of the user.

3. The apparatus according to claim 1, wherein the band comprises a plurality of spaced apart and substantially parallel bands.

4. The apparatus of claim 1, wherein the pillow comprises an outer layer and a padded material arranged within the outer layer.

5. The apparatus of claim 4, wherein the band and the outer layer of the pillow are each made of a cotton material.

6. The apparatus of claim 1, wherein the band has a length of approximately twelve inches and the pillow has a length of approximately eleven inches, and wherein the passage is formed perpendicularly to the length of the pillow.

7. The apparatus of claim 6, wherein a height of the pillow is approximately nineteen inches, and a width of the pillow is approximately six inches.

8. The apparatus of claim 1, wherein the pillow comprises two opposing edges and each of two ends of the bands are attached to one of the two opposing edges.

9. The apparatus of claim 8, wherein each of the two ends of the band are sewn to one of the two opposing edges of the pillow.

10. The apparatus of claim 8, wherein a first end of the band is sewn to a first opposing edge of the pillow and a second end of the band is attached to a second opposing edge of the pillow by a detachable connection means.

11. The apparatus of claim 10, wherein the detachable connection means comprises one or more of: a button and opening slot to receive the button, a tab and tab receiving arrangement, a zipper mechanism, hook and loop fasteners, a strap and loop configured to receive the strap therethrough with strap securing mechanism, a string, laces or other tying mechanism, a snap and button arrangement, and magnetic connectors.

12. The apparatus of claim 1, wherein the apparatus is configured to be worn by a user during sleep and is configured to warm the user's leg during sleep and increase space between the user's legs during sleep.

13. The apparatus of claim 1, wherein the band of the apparatus is configured to enable the user to rotate the pillow around the leg between different positions.

14. A method for reducing leg pain and heat loss of a user, comprising:
providing a leg pillow to the user, the leg pillow comprising:
a pillow; and
a band having two opposing ends each affixed to the pillow, the band at least partially overlying a surface of the pillow,
wherein the band is configured to be pulled away from the surface of the pillow to provide a passage formed between the band and the surface of the pillow; and
inserting a leg of the user through the passage to secure the pillow to the leg of the user.

15. The method of claim 14, wherein the step of inserting the leg of a user through the passage to secure the pillow is performed prior to the user sleeping, and the pillow is secured to the leg of the user during sleep.
